(19) United States
(12) Patent Application Publication
(10) Pub. No.: US 2010/0028394 A1
(43) Pub. Date: Feb. 4, 2010

(54) COSMETIC COMPOSITION FOR MAKING UP AND/OR CARING FOR THE LIPS

(75) Inventor: Xavier BLIN, Paris (FR)

Correspondence Address: OLIFF & BERRIDGE, PLC, P.O. BOX 320850, ALEXANDRIA, VA, 22320-4850, US

(73) Assignee: L'OREAL, Paris (FR)

(21) Appl. No.: 12/491,852

(22) Filed: Jun. 25, 2009

Related U.S. Application Data
(60) Provisional application No. 61/081,743, filed on Jul. 18, 2008.

(30) Foreign Application Priority Data
Jun. 27, 2008 (FR) 08 54354

(51) Int. Cl. A61K 8/02

(52) U.S. Cl. 424/401

(57) ABSTRACT

The present invention is targeted at a cosmetic composition, in particular in stick form, formed of at least one liquid core comprising at least one fatty phase which is liquid at ambient temperature and held inside a solid sheath comprising at least one fatty substance which is solid at ambient temperature.

It also relates to a cosmetic method for caring for and/or making up keratinous substances which consists in applying, to the latter, by means of a stick, a composition formed of at least one liquid core comprising at least one fatty phase which is liquid at ambient temperature and held inside a solid sheath.

This non provisional application claims the benefit of French Application No. 08 54354 filed on Jun. 27, 2008 and U.S. Provisional Application No. 61/081,743 filed on Jul. 18, 2008.

The present invention relates to a cosmetic composition for making up and/or caring for the lips.

The development of formulations devoted to making up and/or caring for the lips, having satisfactory properties in terms of application, comfort, hold and coverage but also in terms of makeup effect, namely in particular the achievement of gloss and transparency, is an ongoing objective.

The lipstick, which appeared at the beginning of the last century, has today become established and is recognized by users as the most popular method of application in making up their lips. This method of application offers the users a means of choice, in terms of coverage of the lips and of diversity in the colours. However, the users, always on the look out for novel performances on the part of their makeup, also want the latter to be glossy, and significantly so.

Furthermore, it is known that it is possible to conventionally confer a glossy nature by the use, in this type of composition, of viscous oils with a high refractive index, such as diisostearyl malate, and/or pasty compounds, such as diglyceryl polyacyl adipate. However, the use of these compounds then generally presents problems of stickiness and migration. The addition of fillers can certainly compensate for the migration but unfortunately detrimentally affects the gloss of the compositions obtained.

For all these reasons, the formulations which generally provide this effect of gloss correspond to fluid formulations of gloss type, also more particularly known as “liquid gloss” or “lip gloss”.

However, such formulations are generally not able to jointly provide the same properties of comfort of application, of coverage and of hold as those provided by stick formulation forms. In addition, these soft formulations cannot be made up into a stick without losing their properties of transparency and of gloss. This is because the structuring as a stick does not make it possible to retain the liquid or semiliquid nature and requires the addition of waxes which make possible this structuring. In point of fact, these waxes have a significant detrimental effect on the gloss and transparency of the mixture.

Consequently, there remains to this day a need to have available a cosmetic composition for making up and/or caring for the lips which reproduces the makeup qualities of a stick in terms of ease of application, of coverage and of hold and which furthermore has the qualities of intense gloss and/or transparency of the glosses.

The present invention is specifically targeted at meeting this need.

The inventors have found that a formulation exhibiting a two-fold architecture, namely a solid sheath with a texture similar to that of a stick surrounding a liquid formulation of gloss type, was satisfactory in these terms.

Thus, the subject-matter of the present invention is mainly a cosmetic composition, in particular in stick form, formed of at least one liquid core comprising at least one fatty phase which is liquid at ambient temperature and held inside a solid sheath comprising at least one fatty substance which is solid at ambient temperature.

Thus, the invention relates more particularly to sticks having a construction of the core-sheath type which comprises two different compositions arranged in core-sheath relationship along the longitudinal axis thereof.

Thereby, structures of compositions of the invention are distinct from those of capsules in which a first composition coats the integraly of a second composition being the core of the capsules.

According to the invention, the term “solid” characterizes the state of a composition at ambient temperature and at atmospheric pressure (760 mm Hg).

Preferably the composition according to the invention, when it is solid (part corresponding to the solid sheath of the composition), can be characterized by a value of hardness which can be measured according to the “cheese wire” method well known to a person skilled in the art and described better below in the text.

Within the meaning of the present invention, “liquid” or “fluid”, in contrast to “solid”, is intended to describe any composition capable of matching the shape of its container at ambient temperature.

More specifically, the composition according to the invention, when it is liquid (part corresponding to the liquid core of the composition), can be provided in the form of a fluid paste which can in particular be characterized by a value of viscosity at ambient temperature.

“Ambient temperature” is understood to mean the temperature of the surroundings in which a composition is positioned and which can be set, for reproducible experimental conditions, between 20 and 25° C.

Contrary to all expectations, the inventors have succeeded in developing a cosmetic composition architecture comprising these two different types of texture, each forming two separate phases in contact with one another.

More specifically, the appropriate choice of the viscosity of the liquid core, coupled to appropriate dimensioning of the central tube (diameter) comprising the liquid composition relative to the diameter of the solid sheath, makes it possible to ensure maintenance, in particular by capillary action, of the liquid core at the centre of the solid sheath. Thus, for example, for a liquid composition with a viscosity at ambient temperature of approximately 200 poises, a diameter of the central part of the stick of approximately 2 mm, for an external diameter of the stick of approximately 12.7 mm, has proved to be satisfactory.

In addition, the sheath has to have a sufficiently solid texture and the liquid core has to have a sufficiently liquid texture in order for the two not to become mixed and thus to respectively retain their intrinsic properties.

The inventors have also found that, in order for the solid sheath and the core not to become mixed, the choice of the fatty phases of the solid sheath and of the liquid core may prove to be determining. Specifically, the fatty phases of the solid sheath and of the liquid core can advantageously be chosen so that they are incompatible, that is to say that they are immiscible with one another.

The immiscibility of the solid sheath and of the liquid core can thus be the result of a natural incompatibility between the various phases and in particular between the various fatty phases.

Alternatively, it is possible to envisage a composition according to the invention comprising one or more additional phases located specifically between the liquid core and the solid sheath in order to guarantee the immiscibility of the liquid core and of the solid sheath.

A composition in accordance with the present invention exhibits the advantage of reconciling, in one and the same formulation, the properties of gloss and of transparency of a composition of gloss type and the properties of coverage, colour and hold of a lipstick.

Preferably, the present invention is targeted at a composition as defined above in which the liquid core is held inside the solid sheath by capillary action.

Advantageously, in a composition according to the present invention, the fatty phases of the solid sheath and of the liquid core are incompatible and immiscible with one another.

Preferably, the present invention is targeted at cosmetic compositions as defined above, the liquid core of which additionally comprises particles of pyrogenic silica.

According to a specific embodiment, the invention relates to a composition according to the invention in which the solid sheath is structured by at least one wax.

The liquid core like the solid sheath in accordance with the invention, each comprise a physiologically acceptable medium.

“Physiologically acceptable medium” is intended to denote a medium which is particularly suitable for the application of a composition according to the invention to the lips.

According to one embodiment, the compositions in accordance with the invention can be anhydrous.

Anhydrous composition is understood to mean a composition comprising less than 10% by weight, in particular less than 5% by weight, in particular less than 3% by weight, in particular less than 2% by weight and more particularly less than 1% by weight of water, with respect to the weight of the composition, represented by the liquid core or the solid sheath, as the case may be. Advantageously, the composition according to the invention is anhydrous.

According to another aspect, the present invention relates to a cosmetic method for caring for and/or making up keratinous substances, in particular the lips, which consists in applying, to the latter, by means of a stick, a composition formed of at least one liquid core comprising at least one fatty phase which is liquid at ambient temperature and held inside a solid sheath comprising at least one fatty substance which is solid at ambient temperature.

A further subject-matter of the present invention is a cosmetic method in which the composition as defined above is in the form of a stick.

“Keratinous substance” is understood to mean the skin, nails, hair, eyelashes, eyebrows or lips. More particularly, “keratinous substance” denotes the lips.

Liquid Core

As specified above, the core is fluid, in contrast to the sheath coating it, which is solid.

This fluidity can be characterized in terms of viscosity.

The liquid core according to the present invention can have a viscosity varying from 50 to 400 poises and preferably from 80 to 250 poises. This viscosity is measured using a Contraves TV® rotary viscosimeter, equipped with an “MS-r4” spindle, at a frequency of 60 Hz.

The liquid core according to the invention comprises at least one liquid fatty phase comprising at least one fatty substance which is liquid at ambient temperature and at atmospheric pressure.

Liquid Fatty Phase

The fatty phase can be continuous. In this case, it comprises less than 5% of water, in particular less than 1% of water, with respect to its total weight and in particular can be in the anhydrous form.

The fatty phase of the formulation of the liquid core according to the invention can in particular comprise, as liquid fatty substance, at least one volatile or nonvolatile oil or their mixtures.

Volatile Oils

Within the meaning of the present invention, “volatile oil” is understood to mean an oil (or non-aqueous medium) capable of evaporating on contact with the skin in less than one hour at ambient temperature and at atmospheric pressure. The volatile oil is a volatile cosmetic oil which is liquid at ambient temperature and which has in particular a non-zero vapour pressure at ambient temperature and atmospheric pressure, especially a vapour pressure ranging from 0.13 Pa to 40 000 Pa (10⁻³ to 300 mmHg), in particular ranging from 1.3 Pa to 13 000 Pa (0.01 to 100 mmHg) and more particularly ranging from 1.3 Pa to 1300 Pa (0.0 to 10 mmHg).

The volatile hydrocarbon oils can be chosen from hydrocarbon oils having from 8 to 16 carbon atoms and in particular branched C₈-C₁₆ alkanes (also referred to as isoparaffins), such as isododecane (also referred to as 2,2,4,4,6-pentamethylheptane), isodecane, isohexadecane and, for example, the oils sold under the Isopar® or Permethyl® trade names.

Use may also be made, as volatile oils, of volatile silicone oils, such as, for example, volatile linear or cyclic silicone oils, in particular those having a viscosity ≦8 centistokes (8×10⁻⁶ m²/s) and having in particular from 2 to 10 silicon atoms, especially from 2 to 7 silicon atoms, these silicones optionally comprising alkyl or alkoxy groups having from 1 to 10 carbon atoms. Mention may in particular be made, as volatile silicone oil which can be used in the invention, of dimethicones with viscosities of 5 and 6 cSt, octamethylcyclotetrasiloxane, decamethylcyclopentasiloxane, dodecamethylcyclohexasiloxane, heptamethylhexyltrisiloxane, heptamethyloctyltrisiloxane, hexamethyldisiloxane, octamethyltrisiloxane, decamethyltetrasiloxane, dodecamethylpentasiloxane and their mixtures.

Use may also be made of volatile fluorinated oils, such as nonafluoromethoxybutane or perfluoromethylcyclopentane, and their mixtures.

It is also possible to use a mixture of the abovementioned oils.

A liquid core formulation according to the invention can comprise at least one volatile oil in a content varying from approximately 0.1% to approximately 50% by weight, in particular varying from approximately 1% to approximately 40% by weight and more particularly varying from approximately 2% to approximately 25% by weight of volatile oil, with respect to the weight of the liquid core formulation.

Advantageously, the liquid core comprises less than 5% by weight of volatile oil, with respect to the total weight of the liquid core formulation, indeed even does not comprise any of it at all.

Nonvolatile Oils

Within the meaning of the present invention, “nonvolatile oil” is understood to mean an oil having a vapour pressure of less than 0.13 Pa and in particular oils of high molar mass. The nonvolatile oils can be hydrocarbon oils, in particular of vegetable origin, oils of synthetic or mineral origin, silicone oils, fluorinated oils or their mixtures.

Mention may in particular be made, as nonvolatile hydrocarbon oil, of:
nonpolar hydrocarbon oils,
nonpolar oils of high molar mass having in particular a molar mass ranging from approximately 400 to approximately 10 000 g/mol, in particular from approximately 650 to approximately 10 000 g/mol, in particular from approximately 750 to approximately 7500 g/mol, and more particularly varying from approximately 1000 to approximately 5000 g/mol. Mention may in particular be made, as oil of high molar mass which can be used in the present invention, of the oils chosen from:
lipophilic polymers,
nonpolar oils of vegetable origin,
and their mixtures,
   and their mixtures.

For example, an oil of high molar mass can be chosen from lipophilic polymers, such as:
polybutylenes, such as Indopol H-100® (with a molar mass MW=965 g/mol), Indopol H-300® (MW=1340 g/mol) or Indopol H-1500® (MW=2160 g/mol), sold and manufactured by Amoco,
polyisobutylenes, for example hydrogenated polyisobutylenes, such as Panalane H-300 E®, sold or manufactured by Amoco (MW=1340 g/mol), Viseal 20000®, sold or manufactured by Synteal (MW=6000 g/mol), Rewopal PIB 1000®, sold or manufactured by Witco (MW=1000 g/mol), or Parleam® from NOF,
polydecenes and hydrogenated polydecenes, such as: Puresyn 10® (MW=723 g/mol) or Puresyn 150® (MW=9200 g/mol), sold or manufactured by Mobil Chemicals, and
mixtures of these.

The nonvolatile silicone oils which can be used according to the invention can be nonvolatile polydimethylsiloxanes (PDMSs), polydimethylsiloxanes comprising pendant alkyl or alkoxy groups and/or alkyl or alkoxy groups at the ends of the silicone chain, which groups each have from 2 to 24 carbon atoms, phenylated silicones, such as phenyl trimethicone, phenyl dimethicone or diphenyl dimethicone, polydimethylsiloxanes with a viscosity of less than or equal to 100 cSt, and their mixtures.

Mention may also be made, as other polar oils which can be employed according to the invention, of:
polar hydrocarbon oils of vegetable origin, such as phytosteryl esters, for example phytosteryl oleate, phytosteryl isostearate and lauroyl/octyldodecyl/phytosteryl glutamate (Ajinomoto, Eldew PS203), triglycerides composed of esters of fatty acids and of glycerol, the fatty acids of which can in particular have varied chain lengths from C₄ to C₃₆ and in particular from C₁₈ to C₃₆, it being possible for these oils to be linear or branched and saturated or unsaturated; these oils can in particular be heptanoic or octanoic triglycerides; wheat germ, sunflower, grape seed, sesame, maize, apricot, castor, shea, avocado, olive, soybean, sweet almond, palm, rapeseed, cottonseed, hazelnut, macadamia, jojoba, alfalfa, poppy, pumpkinseed, cucumber, blackcurrant seed, evening primrose, millet, barley, quinoa, rye, safflower, candlenut, passionflower or musk rose oil; shea butter; or triglycerides of caprylic/capric acids, such as those sold by Stéarineries Dubois or those sold under the names Miglyol 810®, 812® and 818® by Dynamit Nobel,
synthetic ethers having from 10 to 40 carbon atoms;
the vinylpyrrolidone/1-hexadecene oligomer, Antaron V-216, sold or manufactured by ISP (MW=7300 g/mol),
synthetic esters, such as oils of formula R′₁COOR′₂ in which R′₁ represents a residue of a linear or branched fatty acid comprising from 1 to 40 carbon atoms and R′₂ represents a hydrocarbon chain, in particular a branched hydrocarbon chain, comprising from 1 to 40 carbon atoms, provided that R′₁+R′₂≧10. The esters can in particular be chosen from esters, in particular of fatty alcohol and of fatty acid, such as, for example:
cetearyl octanoate, esters of isopropyl alcohol, such as isopropyl myristate or isopropyl palmitate, ethyl palmitate, 2-ethylhexyl palmitate, isopropyl stearate or isostearate, isostearyl isostearate, octyl stearate, diisopropyl adipate, heptanoates and in particular isostearyl heptanoate, octanoates, decanoates or ricinoleates of alcohols or of polyalcohols, such as propylene glycol dioctanoate, cetyl octanoate, tridecyl octanoate, 2-ethylhexyl palmitate and 4-diheptanoate, alkyl benzoate, polyethylene glycol diheptanoate, propylene glycol di(2-ethylhexanoate) and their mixtures, C₁₂ to C₁₅ alkyl benzoates, hexyl laurate, esters of neopentanoic acid, such as isodecyl neopentanoate, isotridecyl neopentanoate, isostearyl neopentanoate or octyldodecyl neopentanoate, or esters of isononanoic acid, such as isononyl isononanoate, isotridecyl isononanoate or octyl isononanoate;
esters of polyols and esters of pentaerythritol, such as dipentaerythritol tetrahydroxystearate/tetraisostearate,
higher C₁₂ to C₂₂ fatty acids, such as oleic acid, linoleic acid, linolenic acid and their mixtures,
dialkyl carbonates, it being possible for the 2 alkyl chains to be identical or different, such as dicaprylyl carbonate, sold under the name Cetiol CC® by Cognis,
polar oils of high molar mass having in particular a molar mass varying from approximately 400 to approximately 10 000 g/mol, in particular from approximately 650 to approximately 10 000 g/mol, in particular from approximately 750 to approximately 7500 g/mol and more particular varying from approximately 1000 to approximately 5000 g/mol.

Advantageously, the liquid core of a composition according to the invention comprises at least one ester of polyol(s) and of dimer diacid of fatty acid, or one of its esters.

Mention may in particular be made, as polar oil of high molar mass which can be used in the present invention, of the oils chosen from:
linear fatty acid esters having a total carbon number ranging from 35 to 70,
aromatic esters,
branched C₂₄-C₂₈ fatty acid or fatty alcohol esters,
and their mixtures.

For example, a polar oil of high molar mass can be chosen from esters, such as:
aromatic esters, such as tridecyl trimellitate (MW=757 g/mol), such as Liponate TDM® from Lipo Chemicals,
branched C₂₄-C₂₈ fatty acid or fatty alcohol esters, such as those described in Application EP-A-0 955 039, and pentaerythritol esters, and in particular triisoarachidyl citrate (MW=1033.76 g/mol), pentaerythrityl tetraisononanoate (MW=697 g/mol), glyceryl triisostearate (MW=891 g/mol), glyceryl tri(2-decyltetradecanoate) (MW=1143 g/mol), pentaerythrityl tetraisostearate (MW=1202 g/mol), polyglyceryl-2 tetraisostearate (MW=1232 g/mol) or pentaerythrityl tetra(2-decyltetradecanoate) (MW=1538 g/mol),
esters and polyesters of dimer diol, such as esters of dimer diol and fatty acid, and esters of dimer diol and of dimer diacid, such as Lusplan DD-DA5® and Lusplan DD-DA7®, sold by Nippon Fine Chemical and described in Application US 2004-175338, the content of which is incorporated in the present patent application by way of reference.

According to one embodiment of the invention, the liquid core of the composition according to the present invention comprises at least one nonvolatile oil chosen from polybutenes, hydrogenated polyisobutylenes, polydecenes, hydrogenated polydecenes, silicone oils, the vinylpyrrolidone/1-hexadecene oligomer, isononyl isononanoate, esters of polyol(s) and of dimer diacid of fatty acid or one of its esters, aromatic esters, such as tridecyl trimellitate, and their mixtures.

The nonvolatile oils can be present in a liquid core formulation according to the invention in a content varying from 5% to 99% by weight, in particular from 15% to 90% by weight and especially from 30% to 80% by weight, with respect to the total weight of the liquid core formulation.

Other Fatty Substances

Other fatty substances can be employed in the liquid core formulation according to the invention, provided that their nature and/or their amount does not affect the properties of viscosity and of gloss of the liquid core formulation.

Thus, a liquid formulation according to the invention can also comprise at least one fatty substance chosen from waxes, pasty compounds, structuring and/or thickening agents, and their mixtures, as defined for the solid fatty phase of the solid sheath below.

However, the liquid core generally comprises at most half as much of waxes, pasty compounds or structuring and/or thickening agents as the solid sheath. According to a preferred embodiment, the content of these compounds in a liquid core formulation in accordance with the invention is less than 15% by weight, more particularly less than 10% by weight, indeed even less than 5% by weight, with respect to the total weight of the liquid core formulation.

Advantageously, the liquid core can be devoid of wax and/or of pasty compound(s) and/or of structuring and/or thickening compounds.

Advantageously, the said liquid fatty phase can additionally comprise particles of pyrogenic silica.

Pyrogenic Silica

The particles of pyrogenic silica suitable for the implementation of the invention can be hydrophilic or can be surface treated in order to be rendered hydrophobic.

The hydrophilic pyrogenic silicas can be obtained by high-temperature hydrolysis of a volatile silicon compound in an oxyhydrogen flame, producing a finely divided silica. The hydrophilic silicas can exhibit a large number of silanol groups at their surface.

Such hydrophilic silicas are, for example, sold under the names “Aerosil 130®”, “Aerosil 200®”, “Aerosil 255®”, “Aerosil 300®”, “Aerosil 380®” or “Aerosil 972®” by Degussa or “Cab-O-Sil HS-5®”, “Cab-O-Sil EH-5®”, “Cab-O-Sil LM-130®”, “Cab-O-Sil MS-55®” or “Cab-O-Sil M-5®” by Cabot.

The hydrophobic pyrogenic silicas can be obtained by modification of the surface of the silica by chemical reaction which brings about a reduction in the number of silanol groups, it being possible for these groups to be replaced in particular by hydrophobic groups.

The hydrophobic groups can be:
trimethylsiloxyl groups, which are obtained in particular by treatment of pyrogenic silica in the presence of hexamethyldisilazane. Silicas thus treated are named “Silica silylate” according to the CTFA (6^{th} edition, 1995). They are sold, for example, under the references “Aerosil R812®” by Degussa and “Cab-O-Sil TS-530®” by Cabot.
dimethylsilyloxyl or polydimethylsiloxane groups, which are obtained in particular by treatment of pyrogenic silica in the presence of polydimethylsiloxane or of dimethyldichlorosilane. Silicas thus treated are named “Silica dimethyl silylate” according to CTFA (6^{th} edition, 1995). They are sold, for example, under the references “Aerosil R972®” and “Aerosil R974®” by Degussa and “Cab-O-Sil TS-610 ®” and “Cab-O-Sil TS-720®” by Cabot.

Particles of pyrogenic silica can be present in the liquid core formulation in accordance with the invention in a content varying from approximately 1% to approximately 30% by weight, in particular from approximately 2% to approximately 20% by weight and more particularly from approximately 5% to approximately 15% by weight, with respect to the total weight of the liquid core formulation.

The silica provides the formulation comprising it with a thick, fondant and very glossy texture.

Solid Sheath

As specified above, when the composition is in the solid state and forms a solid sheath at ambient temperature, it can be characterized in terms of hardness.

More specifically, as indicated above, in order to determine the hardness of the solid sheath, the “cheese wire” method is used on a sample of the solid sheath. For this, a sample of the solid sheath is hot cast in a mould for sticks with a diameter of 12.7 mm. The mould is subsequently cooled in a freezer for approximately one hour. The lipstick stick obtained is subsequently stored at 20° C.

The hardness of the samples is measured after waiting for 24 hours. The method used consists in transversely cutting the cylindrical stick with a diameter of 12.7 mm obtained above using a stiff tungsten wire with a diameter 250 μm advancing at a rate of 100 mm/min.

The hardness of the samples of the solid sheath according to the invention corresponds to the maximum shear force exerted by the wire on the stick at 20° C., expressed in grams, and is measured using a DFGS2® tensile testing device sold by Indelco-Chatillon.

According to this method, the hardness of the solid sheath in accordance with the invention is between 30 and 500 g, in particular between 100 and 350 g, indeed even between 100 and 300 g.

The solid sheath according to the present invention has a radial thickness of between 2 and 10 mm and preferably of between 3 and 9 mm.

The solid sheath surrounding the liquid core is formed, in all or part, of a fatty phase comprising at least one fatty substance which is solid at ambient temperature and at atmospheric pressure and which can be chosen from pasty compounds, waxes, structuring or thickening agents and their mixtures.

Pasty Compound

The solid sheath can comprise a solid fatty substance, such as at least one pasty compound.

“Pasty” is understood to mean, within the meaning of the present invention, a lipophilic fatty compound which exhibits a reversible solid/liquid change in state, which exhibits, in the solid state, an anisotropic crystalline arrangement and which comprises, at a temperature of 23° C., a liquid fraction and a solid fraction.

The pasty compound is preferably chosen from synthetic compounds and compounds of vegetable origin. A pasty compound can be obtained by synthesis from starting materials of vegetable origin.

The pasty compound can advantageously be chosen from:
lanolin and its derivatives,
polymeric or nonpolymeric silicone compounds,
polymeric or nonpolymeric fluorinated compounds,
vinyl polymers, in particular:
   olefin homopolymers,
   olefin copolymers,
   hydrogenated diene homopolymers and copolymers,
   linear or branched and homo- or copolymeric oligomers of alkyl (meth)acrylates preferably having a C₈-C₃₀ alkyl group,
   homo- and copolymeric oligomers of vinyl esters having C₈-C₃₀ alkyl groups, and
   homo- and copolymeric oligomers of vinyl ethers having C₈-C₃₀ alkyl groups,
fat-soluble polyethers resulting from polyetherification between one or more C₂-C₁₀₀, preferably C₂-C₅₀, diols,
esters,
and their mixtures.

Preference is in particular given, among esters, to:
the esters of an oligomeric glycerol, in particular the esters of diglycerol, especially the condensates of adipic acid and of diglycerol, for which a portion of the hydroxyl groups of the glycerols have reacted with a mixture of fatty acids, such as stearic acid, capric acid, stearic acid and isostearic acid and 12-hydroxystearic acid, such as, in particular, those sold under the Softisan 649® brand by Sasol,
the arachidyl propionate sold under the Waxenol 801® brand by Alzo,
phytosterol esters,
triglycerides of fatty acids and their derivatives,
pentaerythritol esters,
noncrosslinked polyesters resulting from the polycondensation between a linear or branched C₄-C₅₀ dicarboxylic acid or polycarboxylic acid and a C₂-C₅₀ diol or polyol,
aliphatic esters of an ester resulting from the esterification of an aliphatic hydroxycarboxylic acid ester by an aliphatic carboxylic acid,
polyesters resulting from the esterification, by a polycarboxylic acid, of an aliphatic hydroxycarboxylic acid ester, the said ester comprising at least two hydroxyl groups, such as the products Risocast DA-H® and Risocast DA-L®,
esters of dimer diol and dimer diacid, if appropriate esterified on their free alcohol or acid functional group(s) by acid or alcohol radicals, such as Plandool-G®,
and their mixtures.

The choice will preferably be made, among pasty compounds of vegetable origin, of a mixture of soya sterols and of oxyethylenated (5 EO)/oxypropylenated (5 PO) pentaerythritol sold under the reference Lanolide® by Vevy.

The pasty compound can be present in the solid sheath in a content ranging from 0.1 to 50% by weight, in particular ranging from 0.1 to 45% by weight and especially ranging from 0.2 to 30% by weight, with respect to the total weight of the sheath.

The solid sheath can also comprise one or more waxes. Advantageously, it comprises at least one of them.

Waxes

“Wax” is understood to mean, within the meaning of the present invention, a lipophilic fatty compound which is solid at ambient temperature (25° C.), which exhibits a reversible solid/liquid change in state and which has a melting point of greater than or equal to 30° C. which can range up to 200° C. and in particular up to 120° C.

In particular, the waxes suitable for the invention can exhibit a melting point of greater than or equal to 45° C. and in particular of greater than or equal to 55° C.

Within the meaning of the invention, the melting point corresponds to the temperature of the most endothermic peak observed by thermal analysis (DSC) as described in Standard ISO 11357-3; 1999. The melting point of the wax can be measured using a differential scanning calorimeter (DSC), for example the calorimeter sold under the name “MDSC 2920” by TA Instruments.

The measurement protocol is as follows:

A 5 mg sample of wax placed in a crucible is subjected to a first rise in temperature ranging from −20° C. to 100° C. at a heating rate of 10° C./minute, is then cooled from 100° C. to −20° C. at a cooling rate of 10° C./minute and, finally, is subjected to a second rise in temperature ranging from −20° C. to 100° C. at a heating rate of 5° C./minute. During the second rise in temperature, the variation in the difference in power absorbed by the empty crucible and by the crucible comprising the sample of wax is measured as a function of the temperature. The melting point of the compound is the value of the temperature corresponding to the tip of the peak of the curve representing the variation in the difference in power absorbed as a function of the temperature.

The waxes capable of being used according to the invention are chosen from waxes of animal, vegetable mineral or synthetic origin, and their mixtures, which are solid at ambient temperature.

Mention may in particular be made, by way of illustration of the waxes suitable for the invention, of hydrocarbon waxes, such as beeswax, lanolin wax and Chinese insect waxes, rice bran wax, camauba wax, candelilla wax, ouricury wax, esparto wax, berry wax, shellac wax, Japan wax and sumac wax; montan wax, orange and lemon waxes, microcrystalline waxes, paraffin waxes and ozokerite; polyethylene waxes, such as Performalene 500-L polyethylene® from New Phase Technologies, the waxes obtained by the Fischer-Tropsch synthesis and waxy copolymers, and their esters.

Mention may also be made of waxes obtained by catalytic hydrogenation of animal or vegetable oils having linear or branched C₈-C₃₂ fatty chains. Mention may in particular be made, among these, of isomerized jojoba oil, such as the trans-isomerized partially hydrogenated jojoba oil manufactured or sold by Desert Whale under the commercial reference Iso-Jojoba-50®, hydrogenated sunflower oil, hydrogenated castor oil, hydrogenated coconut oil, hydrogenated lanolin oil and di(1,1,1-trimethylolpropane) tetrastearate, sold under the name of Hest 2T-4S® by Heterene.

Mention may also be made of silicone waxes (C₃₀₋₄₅ alkyl dimethicone) or fluorinated waxes.

Use may also be made of the waxes obtained by hydrogenation of castor oil esterified with cetyl alcohol which are sold under the names of Phytowax Castor 16L64® and 22L73® by Sophim. Such waxes are described in Application FR-A-2 792 190.

Use may be made, as wax, of a C₂₀-C₄₀ alkyl (hydroxystearyloxy)stearate (the alkyl group comprising from 20 to 40 carbon atoms), alone or as a mixture.

Such a wax is sold in particular under the names “Kester Wax K 82 P®”, “Hydroxypolyester K 82 P®” and “Kester Wax K 80 P®” by Koster Keunen.

Mention may in particular be made, as microwaxes which can be used according to the invention, of camauba microwaxes, such as that sold under the name of MicroCare 350® by Micro Powders, synthetic wax microwaxes, such as that sold under the name of MicroEase 114S® by Micro Powders, microwaxes composed of a mixture of carnauba wax and of polyethylene wax, such as those sold under the names of MicroCare 300® and 310® by Micro Powders, microwaxes composed of a mixture of carnauba wax and of synthetic wax, such as that sold under the name MicroCare 325® by Micro Powders, polyethylene microwaxes, such as those sold under the names of Micropoly 200®, 220®, 220L® and 250S® by Micro Powders, and polytetrafluoroethylene microwaxes, such as those sold under the names of Microslip 519® and 519 L® by Micro Powders.

The wax can be present in the solid sheath according to the invention in a content of at least 1% by weight, with respect to the total weight of the solid sheath, in particular from 3 to 40% by weight, especially from 5 to 30% by weight, very particularly from 5 to 25% by weight and indeed even from 5 to 20% by weight, with respect to the total weight of the solid sheath.

The solid sheath according to the invention can comprise, in addition to the waxes optionally present, at least one structuring and/or thickening agent chosen from semicrystalline polymers, lipophilic gelling agents and their mixtures.

Semi-Crystalline Polymers

Semi-crystalline polymer is understood to mean compounds comprising at least two repeat units, preferably at least 3 repeat units and more especially at least 10 repeat units. “Semi-crystalline polymer” is understood to mean polymers comprising a crystallizable part, a crystallizable pendant chain or a crystallizable block in the backbone, and an amorphous part in the backbone and exhibiting a first-order reversible phase change temperature, in particular a melting point (solid-liquid transition). When the crystallizable part is in the form of a crystallizable block of the polymer backbone, the amorphous part of the polymer is in the form of an amorphous block; the semi-crystalline polymer is in this case a block copolymer, for example of the diblock, triblock or multiblock type, comprising at least one crystallizable block and at least one amorphous block. “Block” is understood to mean generally at least 5 identical repeat units. The crystallizable block or blocks are then different in chemical nature from the amorphous block or blocks.

The semi-crystalline polymer has a melting point of greater than or equal to 30° C., in particular ranging from 30° C. to 80° C., preferably ranging from 30° C. to 60° C. This melting point is a first-order change in state temperature.

This melting point can be measured by any known method and in particular using a differential scanning calorimeter (DSC).

Advantageously, the semi-crystalline polymer or polymers to which the invention applies exhibit a number-average molecular weight of greater than or equal to 1 000. Advantageously, the semi-crystalline polymer or polymers employed according to the invention have a number-average molecular weight Mn ranging from 2 000 to 800 000, preferably from 3 000 to 500 000, better still from 4 000 to 150 000, in particular less than 100 000, and better still from 4 000 to 99 000. Preferably, they exhibit a number-average molecular weight of greater than 5 600, for example ranging from 5 700 to 99 000. “Crystallizable chain or block” is understood to mean, within the meaning of the invention, a chain or block which, if it were alone, would change reversibly from the amorphous state to the crystalline state according to whether it is above or below the melting point. A chain within the meaning of the invention is a group of atoms which is pendant or lateral with respect to the backbone of the polymer. A block is a group of atoms belonging to the backbone, which group constitutes one of the repeat units of the polymer. Advantageously, the “crystallizable pendant chain” can be a chain comprising at least 6 carbon atoms.

The semi-crystalline polymer can be chosen from block copolymers comprising at least one crystallizable block and at least one amorphous block, homopolymers and copolymers carrying at least one crystallizable side chain per repeat unit, or their blends.

Such polymers are described, for example, in the document EP 1 396 259.

According to a more specific embodiment of the invention, the polymer results from a monomer comprising a crystallizable chain which is chosen from saturated C₁₄ to C₂₂ alkyl (meth)acrylates.

Mention may be made, as specific example of structuring semi-crystalline polymer which can be used according to the invention, of the Intelimer® products from Landec described in the brochure “Intelimer® polymers”, Landec IP22 (Rev. 4-97). These polymers are in the solid form at ambient temperature (25° C.). They carry crystallizable side chains.

Lipophilic Gelling Agents

The gelling agents which can be used according to the invention can be polymeric or molecular and organic or inorganic lipophilic gelling agents.

Mention may be made, as inorganic lipophilic gelling agent, of optionally modified clays, such as hectorites modified by a C₁₀ to C₂₂ ammonium chloride, such as hectorite modified by distearyldimethylammonium chloride, such as, for example, that sold under the name of Bentone 38V® by Elementis.

Mention may also be made of the pyrogenic silica described above in the text.

The polymeric organic lipophilic gelling agents are, for example, partially or completely crosslinked organopolysiloxane elastomers of three-dimensional structure, such as those sold under the names of KSG6®, KSG16® and KSG18® by Shin-Etsu, of Trefil E-505C® and Trefil E-506C® by Dow Corning, of Gransil SR-CYC®, SR DMF10®, SR-DC556®, SR 5CYC gel®, SR DMF 10 gel® and SR DC 556 gel® by Grant Industries, and of SF 1204® and JK 113® by General Electric; ethylcellulose, such as that sold under the name Ethocel® by Dow Chemical; galactomannans comprising from one to six and in particular from two to four hydroxyl groups per monosaccharide which are substituted by a saturated or unsaturated alkyl chain, such as guar gum alkylated by C₁ to C₆ alkyl chains, in particular C₁ to C₃ alkyl chains, and their mixtures; block copolymers of “diblock”, “triblock” or “radial” type of the polystyrene/polyisoprene or polystyrene/polybutadiene type, such as those sold under the name Luvitol HSB® by BASF, of the polystyrene/copoly(ethylene-propylene) type, such as those sold under the name Kraton® by Shell Chemical Co., or of the polystyrene/copoly(ethylene-butylene) type, blends of triblock and radial (star) copolymers in isododecane, such as those sold by Penreco under the name Versagel®, such as, for example, the blend of butylene/ethylene/styrene triblock copolymer and of ethylene/propylene/styrene star copolymer in isododecane (Versagel M 5960).

Mention may also be made, as lipophilic gelling agent, of polymers with a weight-average molecular weight of less than 100 000 comprising a) a polymer backbone having hydrocarbon repeat units provided with at least one heteroatom and optionally b) at least one pendant fatty chain and/or at least one end fatty chain which are optionally functionalized, having from 6 to 120 carbon atoms and being bonded to these hydrocarbon units, such as described in Applications WO-A-02/056847 and WO-A-02/47619; especially polyamide resins (in particular comprising alkyl groups having from 12 to 22 carbon atoms), such as those described in U.S. Pat. No. 5,783,657. Mention may be made, as example of polyamide resin which can be employed according to the present invention, of Uniclear 100 VG®, sold by Arizona Chemical.

Mention may also be made, among the lipophilic gelling agents which can be used according to the invention, of esters of dextrin and of fatty acid, such as dextrin palmitates, in particular such as those sold under the names Rheopearl TL® or Rheopearl KL® by Chiba Flour.

Use may also be made of silicone polyamides of the polyorganosiloxane type, such as those described in the documents U.S. Pat. No. 5,874,069, U.S. Pat. No. 5,919,441, U.S. Pat. No. 6,051,216 and U.S. Pat. No. 5,981,680.

These silicone polymers can belong to the following two families:
polyorganosiloxanes comprising at least two groups capable of establishing hydrogen interactions, these two groups being situated in the chain of the polymer, and/or
polyorganosiloxanes comprising at least two groups capable of establishing hydrogen interactions, these two groups being situated on grafts or branchings.

The solid sheath or phase can additionally comprise other fatty substances, such as the volatile or nonvolatile oils mentioned above, provided that their content and/or their nature do not affect the hardness properties of the solid sheath.

According to a preferred embodiment, the solid sheath/liquid core ratio by weight varies from 0.1 to 5 and preferably from 0.5 to 3.

Advantageously, in a composition according to the invention, the solid sheath has a thickness representing from 10% to 90% of the radius of the stick, more particularly from 20% to 80%, indeed even from 25% to 75%, of the radius of the stick.

Apart from their respective fatty phases, the liquid core like the solid sheath (the combination being defined below as “composition”, unless otherwise indicated) can comprise the additional components normally used in cosmetics, such as colouring materials, fillers or cosmetic active principles.

These additional components are present in amounts adjusted so that the properties of a composition in accordance with the invention are not substantially affected in a detrimental way, in particular in terms of hardness, viscosity and/or gloss.

Colouring Material

A composition in accordance with the present invention can comprise at least one colouring material which can be chosen from water-soluble or water-insoluble, fat-soluble or fat-insoluble and organic or mineral colouring materials, in particular of pigment or pearlescent agent type, conventionally used in cosmetic compositions, materials with an optical effect, and their mixtures.

The colouring materials can be present in a proportion of 0.01 to 40% by weight, preferably of 0.5 to 25% by weight, with respect to the total weight of the composition.

Pigments should be understood as meaning white or coloured and mineral (inorganic) or organic particles which are insoluble in an aqueous solution and which are intended to colour the resulting film.

Mention may be made, as inorganic pigments which can be used in the invention, of titanium, zirconium or cerium oxides, and also of zinc, iron or chromium oxides, ferric blue, manganese violet, ultramarine blue and chromium hydrate.

The pigment can also have a structure which can, for example, be of sericite/brown iron oxide/titanium dioxide/silica type. Such a pigment is sold, for example, under the reference “Coverleaf NS” or “GS” by Chemicals and Catalysts and exhibits a contrast ratio in the region of 30.

The colouring material can also comprise a pigment having a structure which can, for example, be of the type of silica microspheres comprising iron oxide. An example of a pigment exhibiting this structure is that sold by Miyoshi under the reference “PC Ball PC-LL-100 P”, this pigment being composed of silica microspheres comprising yellow iron oxide.

Use may also preferably be made, in compositions according to the invention, of calcium sodium borosilicate in the form of calcium sodium borosilicate platelets coated with titanium dioxide and tin dioxide (94.25/5.25/0.5).

Mention may be made, among the organic pigments which can be used in the invention, of carbon black, pigments of D & C type, lakes based on cochineal carmine of barium, strontium, calcium or aluminium, or the diketopyrrolopyrroles (DPP) described in the documents EP-A-542 669, EP-A-787 730, EP-A-787 731 and WO-A-96/08537.

“Pearlescent agents” should be understood as meaning coloured particles of any shape, which may or may not be iridescent, produced in particular by certain shellfish in their shells or synthesized, which exhibit a colouring effect by optical interference.

The pearlescent agents can be chosen from pearlescent pigments, such as titanium oxide-coated mica covered with iron oxide, titanium oxide-coated mica covered with bismuth oxychloride, titanium oxide-coated mica covered with chromium oxide or titanium oxide-coated mica covered with an organic dye, and pearlescent pigments based on bismuth oxychloride. They can also be mica particles, at the surface of which at least two successive layers of metal oxides and/or of organic colouring materials are superimposed.

Mention may also be made, as examples of pearlescent agents, of natural mica covered with titanium oxide, with iron oxide, with natural pigment or with bismuth oxychloride.

Mention may be made, among the pearlescent agents available on the market, of the “Timica”, “Flamenco” and “Duochrome” (mica-based) pearlescent agents sold by Engelhard, the “Timiron” pearlescent agents sold by Merck, the “Prestige” mica-based pearlescent agents sold by Eckart and the “Sunshine” synthetic mica-based pearlescent agents sold by Sun Chemical.

The pearlescent agents can more particularly have a yellow, pink, red, bronze, orangey, brown, gold and/or coppery colour or glint.

Mention may in particular be made, by way of illustration of the pearlescent agents which can be employed in the context of the present invention, of pearlescent agents of gold colour sold in particular by Engelhard under the name of Brilliant Gold 212G (Timica), Gold 222C (Cloisonne), Sparkle Gold (Timica), Gold 4504 (Chromalite) and Monarch Gold 233X (Cloisonne); bronze pearlescent agents sold in particular by Merck under the names Bronze Fine (17384) (Colorona) and Bronze (17353) (Colorona) and by Engelhard under the name Super Bronze (Cloisonne); orange pearlescent agents sold in particular by Engelhard under the names Orange 363C (Cloisonne) and Orange MCR 101 (Cosmica) and by Merck under the names Passion Orange (Colorona) and Matte Orange (17449) (Microna); brown-coloured pearlescent agents sold in particular by Engelhard under the names Nu Antique Copper 340XB (Cloisonne) and Brown CL4509 (Chromalite); pearlescent agents with a copper glint sold in particular by Engelhard under the name Copper 340A (Timica); pearlescent agents with a red glint sold in particular by Merck under the name Sienna Fine (17386) (Colorona); pearlescent agents with a yellow glint sold in particular by Engelhard under the name Yellow (4502) (Chromalite); red-coloured pearlescent agents with a gold glint sold in particular by Engelhard under the name Sunstone G012 (Gemtone); pink pearlescent agents sold in particular by Engelhard under the name Tan Opale G005 (Gemtone); black pearlescent agents with a gold glint sold in particular by Engelhard under the name Nu Antique Bronze 240 AB (Timica); blue pearlescent agents sold in particular by Merck under the name Matte Blue (17433) (Microna); white pearlescent agents with a silvery glint sold for example by Merck under the name Xirona Silver; and golden green pinkish orangey pearlescent agents sold in particular by Merck under the name Indian Summer (Xirona); and their mixtures.

The cosmetic composition according to the invention can also comprise at least one material with a specific optical effect.

For example, this material can be chosen from particles with a metallic glint, goniochromatic colouring agents, diffracting pigments, thermochromic agents, optical brighteners and fibres, in particular interference fibres.

The particles with a metallic glint which can be used in the invention are chosen in particular from:
particles of at least one metal and/or of at least one metal derivative,
particles comprising an organic or inorganic substrate, made of one or more materials, at least partially covered with at least one layer with a metallic glint comprising at least one metal and/or at least one metal derivative, and
mixtures of the said particles.

Mention may be made, among the metals which can be present in the said particles, for example, of Ag, Au, Cu, Al, Ni, Sn, Mg, Cr, Mo, Ti, Zr, Pt, Va, Rb, W, Zn, Ge, Te, Se and their mixtures or alloys. Ag, Au, Cu, Al, Zn, Ni, Mo, Cr and their mixtures or alloys (for example, bronzes and brasses) are preferred metals.

“Metal derivatives” denotes compounds derived from metals, in particular oxides, fluorides, chlorides and sulphides.

Mention may be made, by way of illustration of these particles, of aluminium particles, such as those sold under the names “Starbrite 1200 EAC®” by Siberline and “Metalure®” by Eckart.

Mention may also be made of metal powders formed of copper or alloy mixtures, such as the references 2844 sold by Radium Bronze, metal pigments, such as aluminium or bronze, for example those sold under the names “Rotosafe 700” from Eckhart, silica-coated aluminium particles sold under the name “Visionaire Bright Silver” from Eckhart and particles formed of metal alloy, such as powders formed of bronze (copper and zinc alloy) coated with silica sold under the name “Visionaire Bright Natural Gold” from Eckart.

The particles can also comprise a glass substrate, such as those sold by Nippon Sheet Glass under the names “Microglass Metashine”.

The goniochromatic colouring agent can be chosen, for example, from interference multilayer structures and liquid crystal colouring agents.

Examples of symmetrical interference multilayer structures which can be used in compositions produced in accordance with the invention are, for example, the following structures: Al/SiO₂/Al/SiO₂/Al, pigments having this structure being sold by DuPont de Nemours; Cr/MgF₂/Al/MgF₂/Cr, pigments having this structure being sold under the name “Chromaflair” by Flex; MoS₂/SiO₂/Al/SiO₂/MoS₂; Fe₂O₃/SiO₂/Al/SiO₂/Fe₂O₃ and Fe₂O₃/SiO₂/Fe₂O₃/SiO₂/Fe₂O₃, pigments having these structures being sold under the name “Sicopearl” by BASF; MoS₂/SiO₂/mica-oxide/SiO₂/MoS₂; Fe₂O₃/SiO₂/mica-oxide/SiO₂/Fe₂O₃; TiO₂/SiO₂/TiO₂ and TiO₂/Al₂O₃/TiO₂; SnO/TiO₂/SiO₂/TiO₂/SnO; Fe₂O₃/SiO₂/Fe₂O₃; SnO/mica/TiO₂/SiO₂/TiO₂/mica/SnO, pigments having these structures being sold under the name “Xirona” by Merck (Darmstadt). By way of examples, these pigments can be pigments with a silica/titanium oxide/tin oxide structure sold under the name “Xirona Magic” by Merck, pigments with a silica/brown iron oxide structure sold under the name “Xirona Indian Summer” by Merck and pigments with a silica/titanium oxide/mica/tin oxide structure sold under the name “Xirona Caribbean Blue” by Merck. Mention may also be made of the “Infinite Colors” pigments from Shiseido. Different effects are obtained according to the thickness and the nature of the various layers. Thus, with the structure Fe₂O₃/SiO₂/Al/SiO₂/Fe₂O₃, the colour changes from green-golden to red-grey for SiO₂ layers of 320 to 350 nm; from red to golden for SiO₂ layers of 380 to 400 nm; from purple to green for SiO₂ layers of 410 to 420 nm; and from copper to red for SiO₂ layers of 430 to 440 nm.

Mention may be made, as examples of pigments with a polymeric multilayer structure, of those sold by 3M under the name “Color Glitter”.

Use may be made, as liquid crystal goniochromatic particles, for example, of those sold by Chenix and of that sold under the name “Helicone® HC” by Wacker.

The fat-soluble dyes are, for example, Sudan red, DC Red 17, DC Green 6, β-carotene, soybean oil, Sudan brown, DC Yellow 11, DC Violet 2, DC Orange 5 or quinoline yellow.

The water-soluble dyes are, for example, beetroot juice or methylene blue.

Fillers

The cosmetic compositions in accordance with the invention can also comprise at least one filler of organic or inorganic nature.

“Filler” should be understood as meaning colourless or white solid particles of any shape which are provided in a form which is insoluble and dispersed in the medium of the composition. Of inorganic or organic nature, they make it possible to confer body or stiffness on the composition and/or softness, mattness and uniformity on the makeup. They are distinct from the particles of pyrogenic silica and colouring materials.

Mention may be made, among the inorganic fillers which can be used in the compositions according to the invention, of talc, mica, silica, trimethylsiloxysilicate, kaolin, bentone, precipitated calcium carbonate, magnesium carbonate, magnesium hydrogencarbonate, hydroxyapatite, boron nitride, hollow silica microspheres (Silica Beads from Maprecos), glass or ceramic microcapsules; Sunsphare L-31 and Sunphare H-31, which are sold by Asahi Glass; Chemicelen, sold by Asahi Chemical; composites of silica and of titanium dioxide, such as the TSG series sold by Nippon Sheet Glass, and their mixtures.

The fillers can in particular be spherical fillers, such as, for example, talc, zinc stearate, mica, kaolin, polyamide (Nylon®) powders (Orgasol® from Atochem), polyethylene powders, tetrafluoroethylene polymer (Teflon®) powders, starch, boron nitride, polymeric microspheres, such as those of poly(vinylidene chloride/acrylonitrile), such as Expancel® (Nobel Industrie) or of acrylic acid copolymers (Polytrap® from Dow Corning), silicone resin microbeads (Tospearls® from Toshiba, for example) and organopolysiloxane elastomers.

Mention may be made, among the organic fillers which can be used in the compositions according to the invention, of polyamide (Nylon®) powders (Orgasol from Atochem), poly-β-alanine powders, polyethylene powders, polytetrafluoroethylene (Teflon®) powders, lauroyllysine, starch, tetrafluoroethylene polymer powders, hollow microspheres formed of polymers, such as Expancel (Nobel Industrie), precipitated calcium carbonate, magnesium carbonate, magnesium hydrogencarbonate, metal soaps derived from organic carboxylic acids having from 8 to 22 carbon atoms, preferably from 12 to 18 carbon atoms, for example zinc stearate, magnesium stearate, lithium stearate, zinc laurate or magnesium myristate, Polypore® L 200 (Chemdal Corporation), silicone resin microbeads (Tospearl® from Toshiba, for example), polyurethane powders, in particular powders formed of crosslinked polyurethane comprising a copolymer, the said copolymer comprising trimethylol hexyllactone. In particular, a hexamethylene diisocyanate/trimethylol hexyllactone copolymer may be involved. Such particles are in particular available commercially, for example under the name of Plastic Powder D-400® or Plastic Powder D-800® from Toshiki, and their mixtures.

A filler can be present in the liquid core according to the invention in a content varying from 0.1 to 10%, preferably from 0.1 to 5% and preferably from 0.5 to 3%, with respect to the weight of the liquid core.

A filler can be present in the solid sheath according to the invention in a content varying from 0.1 to 20%, preferably from 0.5 to 15% and preferably from 1 to 5%, with respect to the weight of the solid sheath.

Advantageously, all the fillers are present in the solid sheath. In particular, the solid sheath comprises at least twice as much filler as the liquid core.

Additional Normal Cosmetic Ingredients

The composition according to the invention can additionally comprise any normal cosmetic ingredient which can be chosen in particular from antioxidants, fragrances, preservatives, neutralizing agents, surfactants, sunscreens, sweeteners, vitamins, moisturizing agents, emollients, hydrophilic or lipophilic active principles, agents for combating free radicals, sequestering agents and their mixtures.

Of course, a person skilled in the art will take care to choose the optional additional ingredients and/or their amounts so that the advantageous properties of the composition according to the invention are not, or not substantially, detrimentally affected by the envisaged addition.

Preparation Process

The liquid core and the solid sheath can be prepared according to known processes commonly used in the cosmetic or dermatological field.

Generally, the composition intended to form the solid sheath is hot cast inside a mould comprising a removable central core with the diameter desired for the liquid part. After cooling, the central core of the mould is removed, via the end of the mould opposite that defining the surface of application, generally bevelled, of the stick, thus giving way to a tube into which a hollow needle is introduced. The composition, after optional heating for the purpose of lowering the viscosity thereof, is then introduced by means of the hollow needle into the tube until the latter is filled. After cooling, the stick is removed from the mould and subsequently packaged in a conventional fashion.

The invention is illustrated in more detail in the following examples, given by way of illustration and without a limiting nature. The percentages are percentages by weight.

### EXAMPLE 1

### Preparation of a Liquid Formulation of the Core

The liquid formulation of the core is produced in accordance with processes well known to a person skilled in the art.

A makeup product of gloss type for the lips is prepared, the composition of which is as follows:

| | % by weight of |
|---|---|
| Ingredient | the liquid core |
| Tridecyl trimellitate (Liponate TDM ® from Lipo | 34.07 |
| Chemicals) | |
| Hydrogenated polyisobutene (Parleam ® from NOF) | 34.07 |
| Polybutene (Indopol H-100 ®) | 20 |
| Diacid Condensate in C₃₆ hydrogenated/ethylenediamine, | 0.4 |
| esterified with stearyl alcohol (molecular weight of | |
| approximately 4000) and stabilized (Uniclear 100 VG ® | |
| from Arizona Chemical) | |
| Hydrophobic pyrogenic silica, surface treated with | 10.5 |
| dimethylsilane (Aerosil R 972 ® from Degussa) | |
| Preservatives | q.s. |
| Fragrance | 0.5 |
| Total | 100 |

The procedure used is as follows.

The polybutene, the polyisobutylene, the tridecyl trimellitate, the polyamide and the preservatives are mixed at the melting point of the polyamide, i.e. approximately 105° C.

Once the mixture is homogeneous and the polyamide fully melted, the fragrance and the particles of pyrogenic silica are gradually added using a Rayneri mixer while maintaining the temperature at 100-105° C.

The mixture is stirred until completely homogeneous and a translucent mixture has been obtained.

For greater transparency, this mixture can be subjected to the Rayneri mixer under vacuum in order to remove possible air bubbles present in the finished product.

The composition is subsequently poured into the solid sheath.

### EXAMPLE 2

### Preparation of a Liquid Formulation of the Core

Tridecyl trimellitate is mixed with isoparaffin (6 to 8 mol of isobutylene), a dilinoleyl diol/dilinoleic copolymer and the polybutene (monoolefin/isoparaffin—85/15) at a temperature of 90° C. Additional fatty substances, such as waxes, more specifically a polyethylene wax, are subsequently added to this mixture at 100° C. The combined product is mixed until homogeneous and then the silica and the pearlescent agent are dispersed using a Rayneri mixer.

The composition of the formulation thus obtained is described in detail below.

| | % by weight of |
|---|---|
| Ingredient | the liquid core |
| Di(tertiobutyl)-4-hydroxytoluene | 0.06 |
| Hydrophobic pyrogenic silica, surface | 4 |
| treated with dimethylsilane | |
| Calcium and sodium borosilicate platelets | 3 |
| coated with titanium dioxide and tin | |
| dioxide (94.25/5.25/0.5) | |
| Tridecyl trimellitate (Liponate TDM ® from | 24.94 |
| Lipo Chemicals) | |
| Fragrance | 0.5 |
| Hydrogenated isoparaffin (6-8 mol of | 26 |
| isobutylene) | |
| Polybutene (Monoolefins/Isoparaffins | 10 |
| (85/15)) (MW: 1290) | |
| Polyethylene wax (MW: 500) | 1.5 |
| Dilinoleyl diol dimer/dilinoleic dimer | 30 |
| copolymer | |
| TOTAL | 100 |

### EXAMPLE 3

### Preparation of the Solid Formulation Constituting the Solid Sheath

The composition of the solid shell is in accordance with that of a conventional stick of lipstick, the latter being chosen in particular for its qualities of application and of comfort.

% by weight of
the solid sheath

Phase A

| | |
|---|---|
| Butylated hydroxytoluene (BHT) | 0.06 |
| PEG-45 copolymer/dodecyl glycol (Elfacos ST9 ® | 6 |
| from Akzo Nobel) | |
| Octyldodecyl neopentanoate | 18 |
| Polybutene | 15 |
| Triisostearin | 7 |
| Octyldodecyl/PPG-3 myristyl ether dilinoleate dimer | 1 |
| Bis-diglyceryl polyacyladipate-2 (Softisan 649 ® from | 15 |
| Sasol) | |
| Isostearyl isostearate | 10 |

Phase B

| | |
|---|---|
| Disteardimonium hectorite (Bentone 38V ® from | 1 |
| Elementis) | |

Phase C

| | |
|---|---|
| Yellow 6 Lake ® | 7 |
| Red 7 ® | 4 |
| Titanium dioxide | 1 |

Phase D

| | |
|---|---|
| Polyethylene wax (Performalene 500-L | 5 |
| polyethylene ® from New | |
| Phase Technologies) | |
| Microcrystalline wax (Microwax HW ® from Paramelt) | 7 |

Phase E

| | |
|---|---|
| Mica | 2.94 |

The ingredients of phase A are added to a heating vessel. The combined mixture is stirred with a Rayneri mixer while heating gently at 50° C.

The pigments of phase C are milled in a portion of phase A.

The Bentone 38V® (phase B) is dispersed in the other portion of phase A.

The milled product, the hectorite dispersed in phase A and the waxes of phase D are added to a jacketed heating vessel. The combined mixture is heated at 98-100° C. while stirring with a Rayneri mixer until the waxes have melted.

Finally, phase E is added to the mixture, which is cast in a mould in order to produce sticks with a diameter of 12.7 mm.

### EXAMPLE 4

### Composition According to the Invention Comprising a Liquid Core and a Solid Shell

Cosmetic compositions according to the invention can be produced by employing the compositions prepared according to Examples 1, 2 and 3. To do this, use is made of a double-walled mould in which the solid composition intended to form the shell is cast first within the outer wall of the mould. In a second stage, the formulation intended to form the liquid core (Example 1 or 2) of the composition is poured within the inner wall of the preceding shell.

1. Cosmetic composition formed of at least one liquid core comprising at least one fatty phase which is liquid at ambient temperature and held inside a solid sheath comprising at least one fatty substance which is solid at ambient temperature.

2. Composition according to claim 1, wherein said composition is in stick form.

3. Composition according to claim 1, wherein the liquid core is held inside the solid sheath by capillary action.

4. Composition according to claim 1, wherein the liquid core comprises at least one nonvolatile oil chosen from polybutenes, hydrogenated polyisobutylenes, polydecenes, hydrogenated polydecenes, silicone oils, the vinylpyrrolidone/1-hexadecene oligomer, isononyl isononanoate, esters of polyol(s) and of dimer diacid of fatty acid or one of its esters, aromatic esters, and their mixtures.

5. Composition according to claim 1, wherein the liquid core comprises at least one ester of polyol(s) and of dimer diacid of fatty acid, or one of its esters.

6. Composition according to claim 1, wherein the liquid core additionally comprises particles of pyrogenic silica.

7. Composition according to claim 6, wherein the particles of pyrogenic silica are present in a content varying from 1% to 30% by weight, with respect to the total weight of the liquid core.

8. Composition according to claim 1, wherein the solid sheath comprises at least one wax.

9. Composition according to claim 8, wherein the wax is present in a content of at least 1% by weight, with respect to the weight of the solid sheath.

10. Composition according to claim 1, wherein the solid sheath has a hardness of between 30 and 500 g.

11. Composition according to claim 1, wherein the viscosity at 25° C. of the liquid core varies from 50 to 400 poises.

12. Composition according to claim 1, wherein the solid sheath/liquid core ratio by weight varies from 0.1 to 5.

13. Composition according to claim 1, wherein the composition additionally comprises at least one colouring material present in a content varying from 0.01 to 40% by weight, with respect to the total weight of the composition.

14. Composition according to claim 13, wherein the colouring material is chosen from water-soluble or water-insoluble, fat-soluble or fat-insoluble and organic or mineral colouring materials.

15. Composition according to claim 1, the composition being anhydrous.

16. Cosmetic method for caring for and/or making up keratinous substances, comprising applying, to the keratinous substances, by means of a stick, a composition as defined according to claim 1.

17. Cosmetic method according to claim 16, in which the composition is in the form of the stick.
